(19) United States
(12) Patent Application Publication
(10) Pub. No.: US 2010/0036429 A1
(43) Pub. Date: Feb. 11, 2010

(54) Plate for fixing bone fragments

(76) Inventor: Alfred Buck, Bondorf (DE)

Correspondence Address: DeMont & Breyer, LLC, 100 Commons Way, Ste. 250, Holmdel, NJ, 07733, US

(21) Appl. No.: 12/534,953

(22) Filed: Aug. 4, 2009

(30) Foreign Application Priority Data
Aug. 11, 2008 (DE) 10 2008 037 204.8

(51) Int. Cl. A61B 17/80; A61F 2/28

(52) U.S. Cl. 606/280; 623/16.11

(57) ABSTRACT

The invention concerns a plate for fixing bone fragments, where the plate consists of a pressed molded body of wire, preferably a pressed knitted fabric.

### FIELD OF INVENTION

The invention concerns a plate for fixing bone fragments and a method for making a plate for fixing bone fragments.

### BACKGROUND OF THE INVENTION

Plates for fixing bone fragments, which are also called bone plates, are known. As a rule these are solid plates in which drillings are made in order to fix bone pieces by means of screws or wires. Such plates are available in many different shapes, in each case according to purpose.

The known bone plates usually consist of metal, in particular surgical stainless steel or titanium. For reliable fixing of bone fragments, especially when dealing with larger fragments like femur fragments, the bone plate must, as a rule, have high strength. Bone plates of metal, as a rule, have sufficiently high strength. However, the high strength is accompanied by very high stiffness so that, as a rule, it is not possible or only just barely possible to deform a bone plate during the corresponding operation so that it precisely matches the curvatures of the bone fragments. To equalize tolerances, bone plates often have oblong holes, so that there is some play between the attachment screw and the bone plate. Such play of course is disadvantageous for the healing process.

In addition, during the healing process bone fragments can move up against each other. In particular, fracture healing and a resulting bony prominence that arises during healing often go hand in hand with slight shifting of the bone fragments relative to each other. In this case traditional bone fragments cannot give way, which for one thing can be disadvantageous for the healing process. For another thing stresses can arise, due to which the screws with which the plate is secured come firmly seated so that at a later point can be very difficult to remove them.

### SUMMARY OF THE INVENTION

In contrast, the invention is based on the task of at least reducing said disadvantages of the known bone plates.

In particular, it is a task of the invention to provide a bone plate that is easy to position and that matches the curvatures of the bone fragments to a certain degree.

Another task of the invention is to provide a bone plate that has high stability but that at the same time allows small shifts of the bone fragments toward each other, for instance as they knit.

Another task of the invention is to be able to provide a stable bone plate with low weight.

The task of the invention is solved by a plate for fixing bone fragments and a method for producing a plate for fixing bone fragments.

The invention moreover concerns an implant of a pressed molded body and an implant that is designed to be bioabsorbable.

The inventor has found that pressed molded bodies of wire are excellently suitable for use as bone plates. Compared to traditional bone plates of steel or titanium, bone plates consisting of a pressed molded body of wire have a certain elasticity, which for one thing allows, during the operation, smaller tolerances, for example curvatures in bone or imprecisely positioned drillings, to be compensated. For another thing, a pressed molded body of wire is at the same time transversely elastic and therefore it can follow slight healing-related shifts of the bone plate.

Thus, the positioning of the bone plates in accordance with the invention is considerably easier, and at the same time stresses do not arise during the healing process.

In addition, a pressed molded body of wire can have very high strength. In particular, a pressed molded body of wire can be made so that its spring characteristic increases with increasing deformation. Such a bone plate does allow slight movements and shifting, but it effectively prevents the drifting of the bone fragments away from each other when there are greater forces.

Another advantage of a bone plate in accordance with the invention is its relatively low weight compared to its strength.

Preferably, the pressed molded body is made of a mesh product knitted material. In this way the strength increases, for one thing, and, for another, no wire tips project out from the body.

In a further development of the invention the knitted material is designed as knitted fabric, preferably as tubular knitted fabric. When pressed together, the meshes of the knitted fabric partially interlock, so that a molded body with very high strength is formed. The inventor further discovered that when a tubular knitted fabric is used, the strength of the molded body is better over its entire volume. If nontubular knitted fabrics are used, the body can be weaker along the edges of the former, as a sheet material formed from knitted material.

Preferably, the pressed molded body is made of a folded or rolled knitted material. In this way the wire or wire mesh is uniformly distributed in the molded body and uniformly interlocked.

In another development of the invention, the plate has at least one recess for insertion of a screw or a wire, where the apparent density of the molded body in the region of the recess, thus around the recess, is higher than in a segment of the molded body that is at a distance from the recess. The recess corresponds to a drilling in a traditional bone plate. The introduction of drillings into a pressed molded body of wire, however, would damage the structure, since the mesh will become distorted in this region. For this reason the recesses are preferably made during the pressing operation, for example by a stamp in the mold. The apparent density of the molded body is the density of the bone plate based on its volume including pores, where the recesses are not to be understood as pores and accordingly the volume of the recesses does not belong to the volume of the molded body.

Higher apparent density in the region of the recesses means that here the material is more densely packed and thus the apparent density is higher. The strength of the molded body is increased in the region of the recesses so that, for one thing, the material loss in the region of the recesses is compensated, and, for another, provision is made for more reliable attachment with screws. In traditional bone plates of solid materials, drillings always mean weak points, in which cracking in particular can occur.

An higher apparent density in the region of recesses can already be achieved by putting a knitted material into a press mold essentially uniformly distributed and pressing it to form a molded body. The recesses in this case are formed by dies in the mold. The material displaced by the dies increases the apparent density in the region of the recesses formed by the dies.

Preferably, the recesses are beveled at the edges so that screw heads can be set flush in the bone plate. Also, the production of bevels is possible already during the pressing process, for example by means of a suitable die.

In another development of the invention, the molded body comprises at least two segments with different apparent density. In particular, it is intended to provide a first segment whose apparent density is at least 3%, preferably at least 10%, and especially preferably at least 20% lower than the apparent density of a second segment.

In doing so it is not only intended to increase the apparent density in the region of the recesses with respect to the average apparent density of the bone plate, but it is also intended in accordance with the invention to provide regions with reduced apparent density. Such regions can, for example, lie in a central segment of the molded body, which then has higher elasticity and in particular can better follow smaller movements of the bone plate during knitting of the bones.

Such a molded body can be provided, for example, by introducing a knitted material inhomogeneously distributed in a mold, thus, a lesser amount of the knitted material is laid in the region in which the molded body is supposed to have reduced apparent density.

The molded body can have porosity between 20 and 80%, preferably between 30 and 70%, and especially preferably between 40 and 60%.

In each case according to purpose plates can be provided with a thickness between 0.5 and 10 mm, preferably between 1 and 8 mm, and especially preferably between 1. 5 and 4 mm.

Thus, a pressed molded body of a knitted material of wire is not necessarily considerably thicker than traditional bone plates of solid material.

In a particular embodiment of the invention the wire consists of titanium or a titanium alloy. It turned out that titanium and titanium alloys grow in especially well. The use of titanium is thus suggested where particularly firm bonding of the bone plate with the surrounding tissue is indicated.

However, it is also conceivable to make the wires of the molded body partly of titanium and partly of another wire material, for example if in-growth is desired, but is desired in a reduced form.

Surgical stainless steel in particular is suitable as a material that does not have the same tendency for in-growth as titanium. As a rule, it has high strength and can be processed easily into a knitted material.

In an alternative embodiment of the invention, the wire consists of a bioabsorbable material, in particular magnesium or a magnesium alloy. Thus, with the invention it is also possible to provide bioabsorbable bone plates that decompose in a defined way.

The decomposition process benefits from the fact that a pressed molded body of wire has high porosity with relatively large open pores. For this reason the decomposition process begins not only on the outside of the molded body, but also within it. In the case of traditional solid bone plates, decomposition processes would begin from the exterior. Probably after some time decomposition would be high enough that in the region of the recesses a reliable hold of the screws would no longer be possible and therefore the bone plate would no longer be effective. Then the decomposition process of the remaining residual material would, however, be drawn out over a much longer period of time.

The mesh size can be 0.1-50 mm, preferably 0.5-20 mm, and especially preferably 3-8 mm.

A plate that is at least segmentally flexible and has an initial spring constant between 50 and 3000 N/mm, preferably between 100 and 1000 N/mm, and especially preferably between 150 and 800 N/mm is provided with the invention.

At the same time, the plate can be seen as a leaf spring, which, if it is fastened at the edges and deflects at the center, can have a spring constant D between 0.1 and 100 N/mm, preferably between 0.5 and 50 N/mm, and especially preferably between 1 and 20 N/mm.

This spring constant preferably refers to the first 1-2 mm of deflection, where the spring constant D increases with increasing deflection, so that the spring plate interpreted as a leaf spring can follow smaller movements, but in the case of larger movement opposes a high resistance so that the bone fragments are reliably held together.

The invention additionally concerns an implant, thus not just a bone plate, but rather, for example, a spinal column implant or a part of a joint socket that comprises a pressed molded body of wire having one or more of the characteristics described above, with the difference that the relevant characteristics refer not to a bone plate, but to an implant in general. In accordance with the invention the wire is at least partially coated. In particular, hard coatings, noble metals like gold, or plastic coatings, in particular PTFE, are envisioned as coatings. Abrasion during movements, which is caused by the wires rubbing against each other, can at least be reduced through such a coating.

In addition, the invention concerns an implant that comprises a pressed molded body of wire, where the wire at least partially consists of a bioabsorbable material, in particular magnesium or a magnesium alloy. In this way a bioabsorbable material can also be used for other areas with the advantage described above.

In another development of the invention, the wire is provided with a bioabsorbable coating. In particular, it is intended that a magnesium wire be coated with a bioabsorbable coating, where the bioabsorbable coating decomposes or dissolves in the body after a certain time and then releases the magnesium wire to decomposition. In this way bioabsorbable implants with a very precisely defined time of use can be provided, where the material first becomes weakened by the decomposition when the coating has dissolved away, and then the decomposition of the implant takes place relatively rapidly.

The invention additionally concerns a method for producing a plate for fixing bone fragments. Here a knitted material is folded and/or rolled up and then pressed into a molded body in a press. A molded body is understood in this case to be a body whose form is essentially predetermined by the press mold, where it is clear that after pressing the body relaxes somewhat and thus does not precisely take the shape of the press mold.

A tubular knitted material is preferably used.

In another development of the invention, the knitted material is folded and/or rolled up so that in at least one area there are fewer plies lying on top of each other than in another area.

In this way a molded body that has an apparent density that varies over the volume can be provided, in particular a molded body can be provided in which the weakened zone is arranged essentially centrally. “Centrally” in the sense of this invention is understood to be a region that is situated between the fastener recesses of the bone plate.

To mold the recesses, a press mold with a die is preferably used. For example, a pin can be inserted into the mold. In the case of larger plates it is even possible to provide, with a single mold, bone plates that have recesses in different positions. In this case the pins could be inserted into different positions in the mold.

Preferably an embossed knitted material is used. In particular one can use a knitted material that has a surface that is corrugated by embossing.

The use of an embossed knitted material increases the tendency of the mesh to hook together with each other during the pressing operation, so that a molded body with higher strength can be provided.

The molded body is preferably molded with a hydraulic or pneumatic press tool. Compared to eccentric presses, for example, these tools have the advantage of deformation that as a rule, is relatively slow, particularly at the end of the press operation. In this way the meshes have more time to hook together during the press operation.

The knitted material can, as is envisioned in another development of the invention, also first be folded and then rolled up.

### BRIEF DESCRIPTION OF THE DRAWINGS

The invention is explained in more detail below with reference to FIGS. 1-4.

FIG. 1 schematically shows an example of a bone plate.

FIG. 2 depicts the folding of a knitted material explained in more detail with reference to the figure.

FIG. 3 depicts the effect of the bone plate as a leaf spring explained in more detail with reference to the figure.

FIG. 4 schematically shows a flow chart with the important process steps for production of a bone plate according to an embodiment example of the invention.

### DETAILED DESCRIPTION

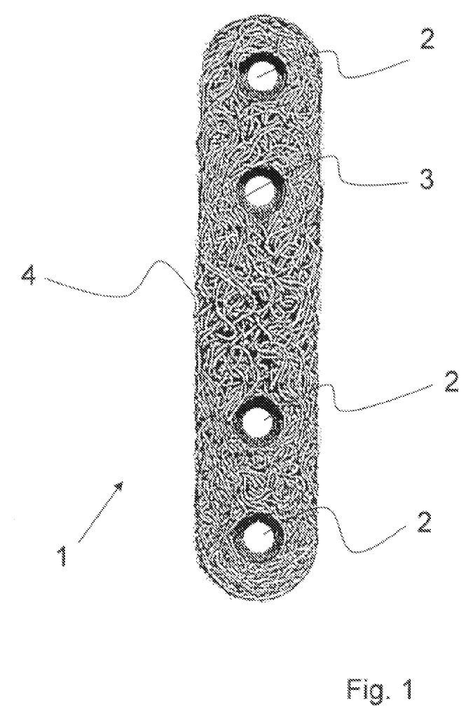

FIG. 1 schematically shows a plate I for fixing bone fragments, also called a bone plate below. Plate 1 consists of a knitted material of wire and in this embodiment example has four recesses 2. The recesses 2 were introduced by means of dies during the press operation and have a bevel 3 for insertion of a screw (not shown).

One can see that the wires around the recess are closer together, and thus the apparent density in the region of the recesses 2 is higher.

In this bone plate, in addition, the knitted material is looser in the central region 4 mainly because fewer plies were laid upon one another. Thus, in the central region 4 the apparent density is again reduced, which leads to higher elasticity in this region.

In this example the bone plate is designed as a simple straight strap for joining two bone fragments.

Of course, the bone plate can have any other shape for other purposes.

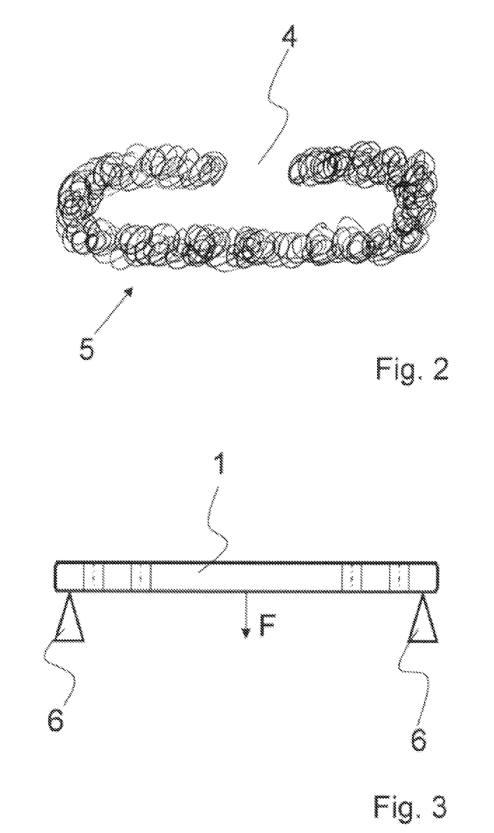

An important step in the production of a bone plate is explained in more detail with reference to FIG. 2.

A knitted material 5 is shown schematically; in this example it is folded once. The knitted material is, however, folded so that no two plies lie on top of each other in the central region 4.

In this way, after the knitted material 5 has been laid into a mold (not shown), a bone plate can be provided whose apparent density is reduced in the central region 4.

A bone plate 1 as a leaf spring is explained in more detail with reference to FIG. 3.

Bone plate 1 is laid on support 6 at its edges. Centrally, a force F is applied to the bone plate vertically downward and the deflection is measured. The initial spring constant D in this case is between 0.5 and 50 N/mm, but it increases with increasing deflection, so that the bone plate allows secure fixing of the bone fragments under greater forces, in spite of its elasticity.

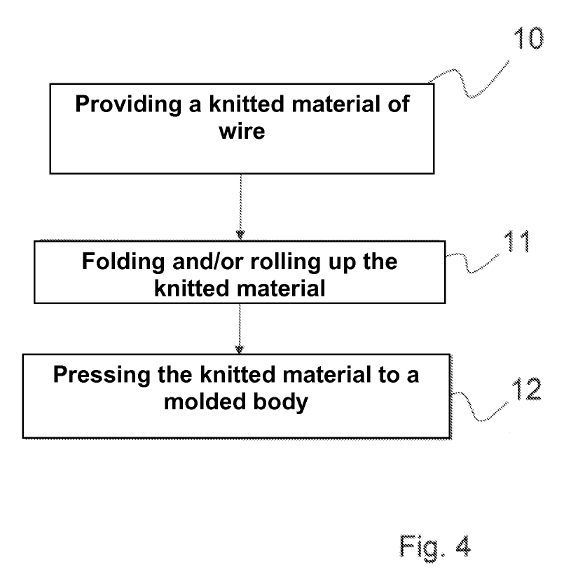

The important steps of a manufacturing process are explained with reference to FIG. 4.

First, a knitted material of wire is provided (10). The knitted material is then folded (11). Alternatively or in addition, the knitted material can also be rolled up.

Then the knitted material is pressed into a molded body (12). The shape of the molded body is essentially given by the shape of the press.

Of course, the invention is not limited to a combination of these characteristics; rather, one skilled in the art will combine all of the characteristics, provided this is meaningful.

### LIST OF REFERENCE NUMBERS

1 Plate
2 Drilling
3 Bevel
4 Central region
5 Knitted material
6 Support
10 Providing knitted material of wire
11 Folding of the knitted material
12 Pressing the knitted material to a molded body

1. A plate for fixing bone fragments, the plate comprising a pressed molded body of wire.

2. A plate for fixing bone fragments as in claim 1, the plate consisting of the pressed molded body of wire.

3. A plate for fixing bone fragments as in claim 1, wherein the pressed molded body is made of a knitted material.

4. A plate for fixing bone fragments as in claim 3, wherein the knitted material is made as a knitted fabric.

5. A plate for fixing bone fragments as in claim 4, wherein the knitted fabric is made as a tubular knitted fabric.

6. A plate for fixing bone fragments as in claim 1, wherein the plate has at least one recess for passage of a screw or a wire, where the apparent density of the molded body in the region of the recess is higher than that of a segment of the molded body at a distance from the recess.

7. A plate for fixing bone fragments as in claim 1, wherein the apparent density of the molded body decreases in the direction of a central region.

8. A plate for fixing of as in claim 1, wherein the plate has at least one recess, where the apparent density of a region surrounding the recess is higher than that of a region further from the recess.

9. A plate for fixing bone fragments as in claim 1, wherein the molded body comprises a first segment, the apparent density of which is at least 10% lower than the apparent density of a second segment.

10. A plate for fixing bone fragments as in claim 1, wherein the molded body has a porosity between 30 and 70%.

11. A plate for fixing bone fragments as in claim 1, wherein the wire consists of titanium or a titanium alloy.

12. A plate for fixing bone fragments as in claim 1, wherein the wire consists of stainless steel.

13. A plate for fixing bone fragments as in claim 1, wherein the wire consists of magnesium or a magnesium alloy.

14. A plate for fixing bone fragments as in claim 1, wherein the molded body consists of a pressed knitted material with a mesh size of 0.5 mm to 20 mm.

15. A plate for fixing bone fragments as in claim 1, wherein the plate is at least segmentally flexible.

16. An implant which has at least one recess for passage of a screw or a wire, the implant consisting of a pressed molded body of wire, where the apparent density of the molded body in the region of the recess is higher than that of a segment of the molded body at a distance from the recess.

17. An implant comprising a pressed molded body of wire, the wire being at least partially coated.

18. An implant comprising a pressed molded body of wire, the wire comprising magnesium or a magnesium alloy.

19. An implant as in claim 18, the wire being provided with a bioabsorbable coating.

20. A method for producing a plate for fixing bone fragments, comprising:
providing a knitted material of wire,
folding and/or rolling up the knitted material, and
pressing the knitted material into a molded body.

21. A method for producing a plate for fixing bone fragments as in claim 21, wherein a tubular knitted material is used.

22. A method for producing a plate for securing bone fragments as in claim 21, wherein the knitted material is folded or rolled up so that, at least in one region, fewer plies are arranged on top of one another than in another region.

23. A method for producing a plate for fixing bone fragments as in claim 21, wherein a press form with at least one die is used to make at least one recess in the plate.

24. A method for producing a plate for fixing bone fragments as in claim 21, wherein an embossed knitted material is used.

25. A method for producing an implant comprising:
providing a knitted material of wire,
folding and/or rolling up the knitted material, and
pressing the knitted material into a molded body, where
a press mold with at least one die is used in order to form at least one recess in the implant.
